(19) United States
(12) Patent Application Publication
(10) Pub. No.: US 2020/0391004 A1
(43) Pub. Date: Dec. 17, 2020

(54) Novel Method and Device for Urinary Catheter Placement

(71) Applicant: MILA INTERNATIONAL, INC., Florence, KY (US)

(72) Inventors: Jeff Vitullo, Pottstown, PA (US); Anita J. Parkin, Jindalee QLD (AU)

(21) Appl. No.: 16/874,108

(22) Filed: May 14, 2020

Related U.S. Application Data
(60) Provisional application No. 62/849,848, filed on May 18, 2019.

(51) Int. Cl. A61M 25/00; A61M 25/01

(52) U.S. Cl. CPC A61M 25/0017; A61M 25/0136; A61M 2205/587; A61M 2210/1092; A61M 2210/1085; A61M 2250/00

(57) ABSTRACT

A new method and device that allows for easy and quick placement of a urinary catheter into the bladder of a female dog whereby only the catheter is left behind.

### FIELD

The present disclosures relate to the medical device and pet industries equally.

### BACKGROUND OF THE DISCLOSURES

Indwelling urinary catheters are regularly placed in hospitalized dogs for the purposes of measuring urine output, inability to urinate, dysuria, stranguria, urinary obstruction, post urinary of bladder surgery and to assist with nursing care in critical care patients^{1 2 3}. It is reported that indwelling urinary catheters are associated with an increased incidence of urinary tract infections². The longer they are in-situ, the higher the chances of acquiring an infection^{3 4 5}, so it is important to make sure that the benefits outweigh the risks⁶, and a sterile technique must be used to minimize contamination to the urinary tract. Catheter-associated urinary tract infections can occur as the catheter is inserted into the bladder¹ and a study showed a 20% incidence of urinary tract infections in female dogs after one-off catheterization.⁴ ¹Silverstein Dombrowski D C. Small Animal Critical Care Medicine. 2009:603-606.² Bexfield N. BSAVA Guide to Procedures in Small Animal Practice. 2 edn, 2014:249, 251.³ Carothers M. Small Animal Internal Medicine for Veterinary Technicians and Nurses. 2012:480-484.⁴ Bubenik U, Hosgood G L, Waldron D R, Snow L A. Frequency of urinary tract infection in catheterized dogs and comparison of bacterial culture and susceptibility testing results for catheterized and noncatheterized dogs with urinary tract infections. J Am Vet Med Assoc 2007;231:893-899.⁵ Bubenik L, Hosgood G. Urinary Tract Infection in Dogs with Thoracolumbar Intervertebral Disc Herniation and Urinary Bladder Dysfunction Managed by Manual Expression, Indwelling Catheterization or Intermittent Catheterization. Veterinary Surgery 2008;37:791-800.⁶ Aldrich J. Advanced Monitoring and Procedures for Small Animal Emergency and Critical Care. 2012:395-406.

Complication that have been reported with indwelling urinary catheters are; trauma to the urethra or urinary bladder and increased risk of urinary tract infections^{2 4 5}.

The goals of urinary catheterization placement are:

Sterile placement of the catheter;

Reduced time for placement;

Smooth entry of the catheter into the urethra with no iatrogenic trauma; and

To empty the bladder fully.

### OBJECTS AND SUMMARY OF THE INVENTION

Techniques:

There are several techniques that have been reported for placing urinary catheters into female dogs. They are further divided into blind and visual techniques. Using the blind technique is when you are unable to visualize the papilla as the urinary catheter enters into the urethra (via palpation). Visual techniques that have been previously reported are by using a speculum (and light source) or otoscope, allowing the papilla to be seen while inserted the urinary catheter^{2 4}.

Equipment Required:

0.05% Chlorhexidine solution flush;

Sterile female urinary catheter device;

Sterile urinary catheter with stylet—to the size of the patient; (See table 1 for recommendations)

Sterile saline or water for injection (to inflate the cuff of the urinary catheter);

Closed urinary collection system;

Sterile lube/Local anaesthetic gel;

Sterile gloves;

Sterile drape;

+/−Light source; and

Elizabethan Collar

TABLE 1
Guide on size of urinary catheter and
device for the weight of the patient.

| | | |
|---|---|---|
| Size of Dog | Foley Size (30 cm) | Size of device |

| | | | | |
|---|---|---|---|---|
| <5 | kg | 5-6 | Fr | X Small |
| 5-10 | kg | 6-8 | Fr | Small |
| 10-20 | kg | 8-10 | Fr | Medium |
| >20 | kg | 10 | Fr | Large |

Preparation:

Gather all the items required before starting to place the catheter. Catheter size guidelines are shown in table 1, however, the largest diameter that will fit with comfort to prevent leakage around the catheter, without causing damage should be chosen³.

Initial preparation of the patient includes clipping a 5 cm area around the vulva (being careful not to damage the skin as this will increase infections)⁶.

Scrub the clipped area with soapy chlorhexidine and then rinse with 0.05% aqueous chlorhexidine. Flush the vagina and vestibule with 0.05% aqueous chlorhexidine five times with 5-10 mls^{3, 6}.

Finish by wiping off the clipped area again with the aqueous chlorhexidine. Exam gloves should be worn for this process.

Put on sterile gloves and have your assistant (if required) also put on gloves. Have the urinary catheter aseptically opened, together with the stylet, sterile lube and devices (speculum, new device, otoscope) if required. Check the balloon before inserted catheter into the patient. Drape the patient with a sterile drape.

Lube the catheter and prepare for the technique of your choice (See different techniques described below).

Once the urinary catheter is in place and placement is confirmed (by having urine exit the catheter), inflate the balloon (to recommended inflation volume that is printed on the catheter). Gently pull on the catheter until the balloon is at the neck of the bladder⁶. Attach a closed urinary catheter collection system to the catheter for urine collection. It is important to maintain a closed urinary collection system to help prevent secondary urinary tract infections3. The collection system must be kept lower than the patient (but not directly on the floor) to help facilitate drainage via gravity and to stop the retrograde of urine to the patient¹. Place an Elizabethan collar on the patient to prevent the catheter being chewed out.

Blind Technique:

The blind technique is performed by inserting a finger into the vulva/vestibule and blocking off the vagina with the finger while feeding the urinary catheter on the midline of the ventral wall of the vestibule into the urethral papilla being conscious not to put too much pressure on the papilla to occlude it^{1,2,6}.

This can be performed in lateral or sternal recumbency and if your patient is compliant, no sedation is required⁶. Using sterile gloves will help prevent iatrogenic urinary tract infections².

Visual Techniques:

The visual technique is performed by inserting the speculum (the opening of the speculum to the ventral wall of the vestibule) while shining a light source (laryngoscope, otoscope or headlight) to visualize the urethral papilla. Pulling gently down (ventrally) on the vulva can help visualize the papilla. The urinary catheter is advanced ventrally within the vestibule, and directed into the urethral papilla. This technique will normally require sedation (or anaesthesia) and can be performed in lateral, dorsal^{1, 2} or sternal recumbency ^{2, 6}. If using an otoscope as the light source the otoscope cone will remain with the patient until the catheter is removed¹. As the catheter is inserted through the cone then is no way to remove it off the catheter once in place⁶.

Modified Visual Technique with Assistance Device (New)

A new technique can be used by inserting a female urinary catheter placement device (FIG. 1) into the vestibule, (the long edge must be on the dorsal wall). This is then advanced into block off the vestibulovaginal junction leaving the short edge on the ventral wall exposing the urethral papilla (FIG. 2). The urinary catheter is then inserted through the centre of the device into the urethral opening. As the vestibulovaginal junction is blocked off with the top of the device there is only one placed the urinary catheter to be advanced into. This technique can be done blindly or by visually seeing the urethral orifice.

As the device protects (encases) the wall of the vulva and vestibule the urinary catheter can be inserted without touching the vestibule wall—hence a more aseptic technique than previously described. This can be done in lateral recumbency and can be done with or without sedation. Once the catheter is in place the device is then removed from the vestibule and taken off the catheter.

Maintenance:

Hand hygiene is very important when maintaining indwelling urinary catheters. Hands must be washed and examination gloves worn before and after touching the patient⁶. Every eight hours (or if the catheter is soiled)^{1, 3} the catheter should be inspected and wiped down with aqueous chiorhexidine 0.05%. The vulva should also be flushed at this point ^{1, 3}. Regular inspections should be checking for flow or urine, irritation of the urinary catheter and urinary output measured and recorded³.

References:

Silverstein Dombrowski DC. Small Animal Critical Care Medicine. 2009:603-606.

Bexfield N. BSAVA Guide to Procedures in Small Animal Practice. 2 edn, 2014:249, 251.

Carothers M. Small Animal Internal Medicine for Veterinary Technicians and Nurses. 2012:480-484.

Bubenik L J, Hosgood G L, Waldron D R, Snow L A. Frequency of urinary tract infection in catheterized dogs and comparison of bacterial culture and susceptibility testing results for catheterized and noncatheterized dogs with urinary tract infections. J Am Vet Med Assoc 2007;231:893-899.

Bubenik L, Hosgood G. Urinary Tract Infection in Dogs with Thorocolumbar Intervertebral Disc Herniation and Urinary Bladder Dysfunction Managed by Manual Expression, Indwelling Catheterization or Intermittent Catheterization. Veterinary Surgery 2008; 37:791-800.

### BRIEF DESCRIPTION OF THE DRAWINGS

Various preferred embodiments are described herein with references to the drawings in which merely illustrative views are offered for consideration, whereby:

FIG. 1 shows Female Urinary Catheter Placement Device;

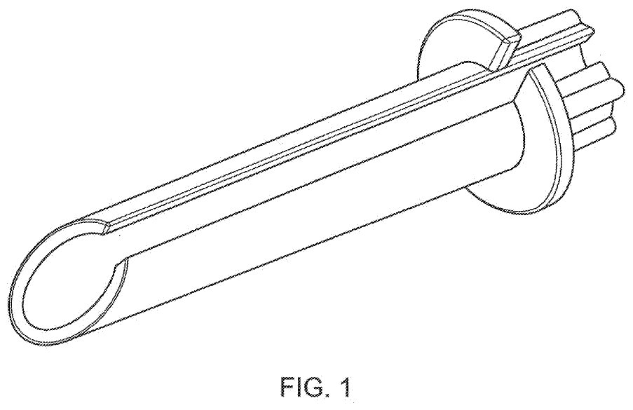

FIG. 2 shows the modified visual technique;

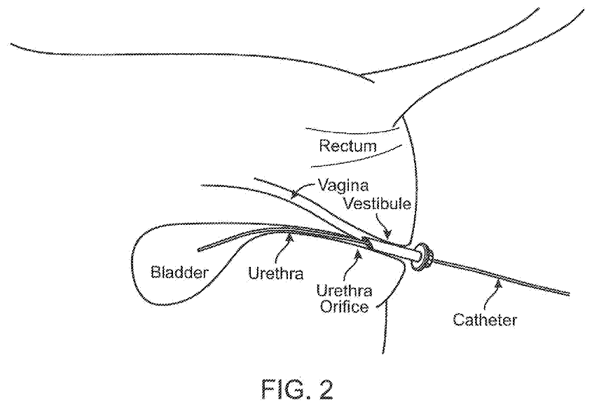

FIG. 3 shows details of the Female Urinary Catheter Placement Device;

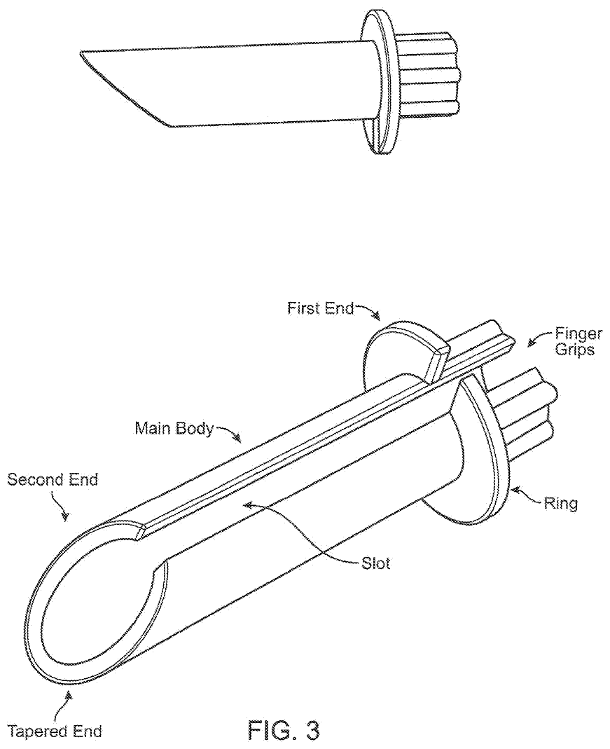

FIG. 4 shows the introducer with Handle and Light Source;

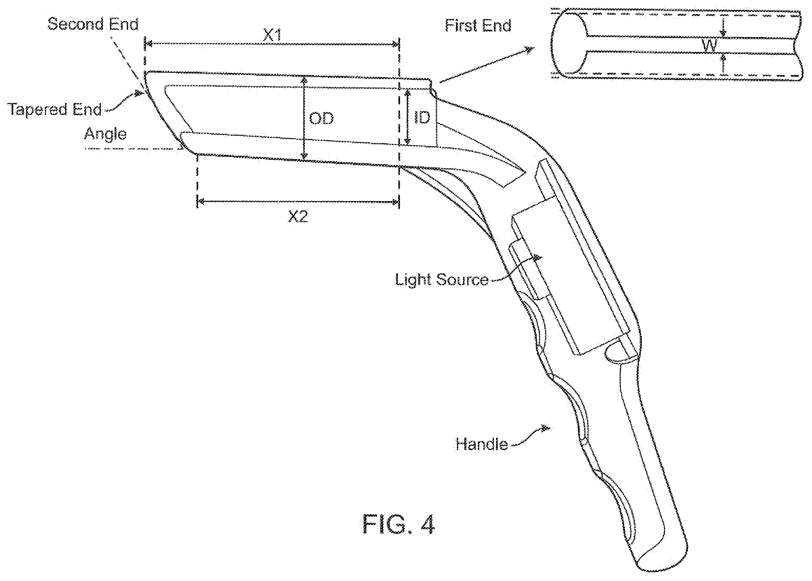

FIG. 5 shows the Light Source.

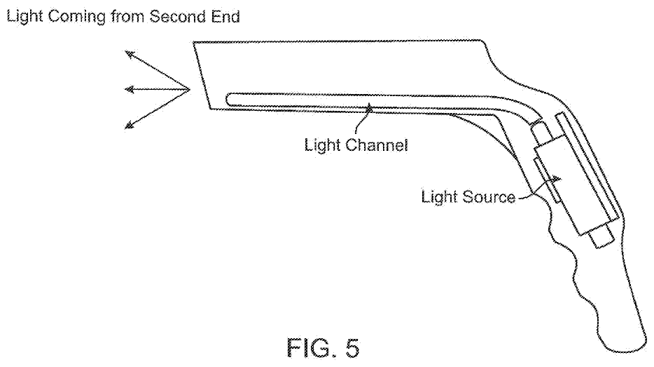

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTIONS

The present inventor has discovered and further invented method and device that allows for easy and quick placement of a urinary catheter into the bladder of a female dog whereby only the catheter is left behind.

Steps are precisely denoued in the claims and their equivalents.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language mans that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of placing a urinary catheter into the bladder of a female dog; comprising: in combination;
a. Position the dog in the standing or in the lateral recumbency position;
b. Gently advance the introducer into the vestibule and against the urethral orifice;
c. Feed the urinary catheter through the introducer and into the urethral orifice. Continue to advance the catheter so that the tip of the catheter resides in the bladder. Urine flow should be observed.
d. Remove the introducer from the dog. Slide the ex-dwelling portion of the urinary catheter body out of the slot in the introducer.

2. A device for introduction of a urinary catheter into the bladder of a female dog; which comprises in combination:
a. A cylindrical main body including a first end and a second end;
b. A hole through the main body, the hole designed to allow a urinary catheter to pass through the main body;
c. A tapered second end to block off the vagina and allow for easy entry into the urethral orifice;
d. A slot along the length of the main body, extending through at least one wall of the main body configured to allow a urinary catheter body to pass through the slot, and,
e. A rim, larger than the cylindrical main body positioned at the first end of the main body configured to prevent one's fingers to advance along the main body towards the second end and between said rim.

3. The method of claim 1, further comprising a slot that is revealed prior to removing the urinary catheter from said introducer.

4. The method of claim 1, wherein the animal is a cat.

5. The method of claim 1, wherein the animal is one of a mammal

6. The method of claim 1, further comprising selecting the introducer based on the breed of the animal.

7. The method of claim 1, further comprising selecting the introducer based on the size of the animal.

8. The method of claim 1, further comprising selecting the introducer based on the type of urinary catheter.

9. The method of claim 1, wherein the urinary catheter and introducer are coupled together during insertion of the introducer.

10. The method of claim 1, wherein the position of the animal is based on the breed of the animal

11. The method of claim 1, wherein the introducer removed from the dog but not removed from the urinary catheter.

12. The method of claim 1, wherein the introducer is split into two halves to break away from the urinary catheter after placement.

13. The device of claim 2, wherein the inner diameter of the main body is smaller than the maximum size of the urinary catheter hub but larger than the outside diameter of the Foley catheter body.

14. The device of claim 2, wherein the urinary catheter is a Foley catheter.

15. The device of claim 2, wherein the channel resides on the opposing side of the taper end.

16. The device of claim 2, wherein the slot shall start at the second end but does not need to end at the first end. It can terminate short of said first end.

17. The device of claim 2, wherein the first end connects to an otoscope for visual placement.

18. The device of claim 2, wherein the tapered end is less stiff than the main body.

19. The device of claim 2, further comprising the first end channels on the surface of the main body to allow one to hold the introducer and alter the angle in order to get into the correct position.

20. The device of claim 2, wherein no rim exists on said main body.

21. The device of claim 2, further comprising a slot that is revealed prior to removing the urinary catheter from said introducer.

22. The device of claim 2, wherein the introducer is constructed out of plastic, metal, or cardboard.

23. The device of claim 2, wherein tapered angle measures between 30 and 60 degrees.

24. The device of claim 2, wherein the first end terminates in a handle.

25. The device of claim 2, wherein the first end terminates in a handle with a light source generating light that travels to the second end.
